(19) United States
(12) Patent Application Publication
(10) Pub. No.: US 2026/0272712 A1
(43) Pub. Date: Sep. 17, 2026

(54) LASER-ADJUSTABLE SHUNTS AND ASSOCIATED SYSTEMS AND METHODS

(71) Applicant: Shitamed Holdings, LLC, Campbell, CA (US)

(72) Inventors: Robert Chang, Belmont, CA (US); Tessa Bronez, Campbell, CA (US); David Batten, San Francisco, CA (US); Tom Saul, Portland, OR (US); Nicholas Lyford, Saratoga, CA (US)

(21) Appl. No.: 19/472,046

(22) PCT Filed: Apr. 19, 2024
(86) PCT No.: PCT/US2024/025489

§ 371 Date: Oct. 2, 2025

Related U.S. Application Data
(60) Provisional application No. 63/497,127, filed on Apr. 19, 2023.
(60) Provisional application No. 63/580,878, filed on Sep. 6, 2023.

(51) Int. Cl. A61F 9/007

(52) U.S. Cl. CPC A61F 9/00781; A61F 2210/0047; A61F 2250/0013

(57) ABSTRACT

The present technology is generally directed to adjustable shunts with actuation assemblies that are configured to be selectively adjusted using laser energy to change a fluid resistance through the shunt. The actuation assemblies include features expected to improve the performance of laser-adjustable shunts, such as by improving the safety profile of laser-adjustable shunts and/or by improving the efficiency of operating laser-adjustable shunts.

### CROSS-REFERENCE TO RELATED APPLICATION(S)

This application claims priority to U.S. Provisional Patent Application No. 63/497,127, filed Apr. 19, 2023, and U.S. Provisional Patent Application No. 63/580,878, filed Sep. 6, 2023, each of which is incorporated by reference herein in its entirety.

### TECHNICAL FIELD

The present technology generally relates to implantable medical devices and, in particular, to shunting systems for promoting fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma shunts or “MIGS”) have shown clinical benefit; however, there is a need for improved shunting systems and techniques for addressing elevated intraocular pressure and risks associated with glaucoma, as well as other patient conditions. For example, there is a need for shunting systems capable of adjusting the therapy provided, including the flow rate/fluid resistance between the two fluidly-connected bodies. As another example, there is a need for shunting systems capable of being modified after manufacture (e.g., in the clinic) to personalize the system for the patient and/or as part of the clinician's plan for the implant procedure.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A illustrates an adjustable shunting system configured in accordance with select embodiments of the present technology.

FIG. 1B is an exploded perspective view of the adjustable shunting system of FIG. 1A.

FIG. 1C is an enlarged exploded view of an actuation assembly of the adjustable shunting system of FIGS. 1A and 1B.

FIG. 1D is an enlarged view of an actuator of the adjustable shunting system shown in FIGS. 1A-1C.

FIG. 2A is an enlarged view of a portion of the adjustable shunting system shown in FIGS. 1A-1C and illustrates an adjustment of the adjustable shunting system in accordance with select embodiments of the present technology.

FIG. 2B is another enlarged view of a portion of the adjustable shunting system shown in FIGS. 1A-1C and illustrates another adjustment of the adjustable shunting system in accordance with select embodiments of the present technology.

FIG. 3A is a perspective view of a portion of the actuator assembly shown in FIG. 1C with select portions omitted for clarity.

FIG. 3B is a cross-sectional view of a portion of the actuator assembly shown in FIG. 3A.

FIG. 4A is an exploded view of another actuation assembly for use with the adjustable shunting system of FIG. 1A and configured in accordance with select embodiments of the present technology.

FIG. 4B is a top view of a plate of the actuation assembly of FIG. 4A.

FIG. 4C is a bottom view of the plate of the actuation assembly of FIG. 4A.

FIG. 5 is a top view of another plate for use with the actuation assembly of FIG. 4A and configured in accordance with select embodiments of the present technology.

FIG. 6A is a partially schematic top view of an actuation assembly having an actuator and a laser energy shield and configured in accordance with select embodiments of the present technology.

FIG. 6B is a partially elevated cross-sectional view of the actuation assembly of FIG. 6A included within a shunting system and configured in accordance with select embodiments of the present technology.

### DETAILED DESCRIPTION

The present technology is generally directed to adjustable shunting systems for promoting the flow of fluid between a first body region and a second body region of a patient. As described throughout this Detailed Description, the adjustable shunting systems can include an actuation assembly for selectively controlling the flow of fluid through the shunting system. For example, the actuation assembly can include one or more actuators that can be selectively and independently actuated to increase or decrease a fluid resistance through the shunting system. In this way, the systems described herein can be selectively titrated to personalize the therapy provided by the system according to individual patient need.

In many embodiments described herein, the actuators are configured to be actuated using laser energy delivered via a laser energy source external to a patient. The systems and actuation assemblies described herein therefore include certain features that are expected to improve the operation of such laser-adjustable shunts, such as by improving the efficiency and/or safety of using laser energy to selectively actuate the shunts after implantation within the patient.

For example, many embodiments of the actuation assemblies described herein include a shape memory actuator and a plate. The actuator can include a first (e.g., “upward facing”) surface and a second (e.g., “downward facing”) surface. The plate can be positioned proximate the second surface of the actuator (e.g., the second surface of the actuator faces the plate and the first surface of the actuator faces away from the plate). When a clinician or other user directs laser energy at the shape memory actuator to drive actuation thereof, some laser energy will be directly absorbed by, and therefore heat, the first surface of the actuator. However, some laser energy will “miss” the upper surface of the actuator. The plate can be positioned to block, absorb, and/or redirect (e.g., reflect) some or all of the portion of the laser energy that misses the first surface. This is expected to be beneficial for a number of reasons. First, the plate acts as a shield or barrier to prevent (or at least reduce) laser energy that misses the first surface of the actuator from penetrating deeper into patient tissue. Second, laser energy that is redirected can subsequently be absorbed by, and thus heat, the second surface of the actuator to assist in driving actuation of the actuator (e.g., the actuator can be simultaneously heated from two opposing surfaces).

In some embodiments, the plate is separated from the second surface of the actuator by a relatively small gap. For example, the gap may be between about 1 μm and about 40 μm, although other dimensions are possible. In certain embodiments, a fluid (e.g., a bodily fluid such as aqueous, blood, etc.) occupies the gap. In such embodiments, some amount of laser energy that is not absorbed by the upper surface of the actuator may be absorbed by, and therefore heat, the plate. The heated plate can (conductively) heat the fluid around the actuator, which can, in turn, (conductively) heat the actuator. Accordingly, heat absorbed by the plate may be transmitted back to the actuator via a transmission medium such as fluid. Accordingly, in some embodiments the plate is expected to heat the fluid in the gap by absorbing some of the laser energy that is not directly absorbed by the actuator. This is expected to be beneficial because it will assist in indirectly heating the actuator via heat transfer from the fluid to the actuator.

In yet other embodiments described herein, the plate is positioned “above” the actuator (e.g., between the laser and the actuator) and acts as an intermediary between the laser energy and the actuator. In such embodiments, the actuator does not directly absorb any laser energy. Rather, a user can direct laser energy toward a particular actuation target on the plate, which can absorb the laser energy as heat. The plate can then conductively transfer the heat to the actuator.

Accordingly, without intending to be bound by theory, the present technology is expected to (1) improve the efficiency with which the actuator can be modulated by the laser energy, and (2) prevent or at least reduce laser energy from penetrating through the system and potentially damaging anatomical structures distal to the shunting system. As will be apparent from the following Detailed Description, the adjustable shunts described herein can include additional features in addition to or in lieu of the foregoing that are also expected to improve the operation of adjustable shunts.

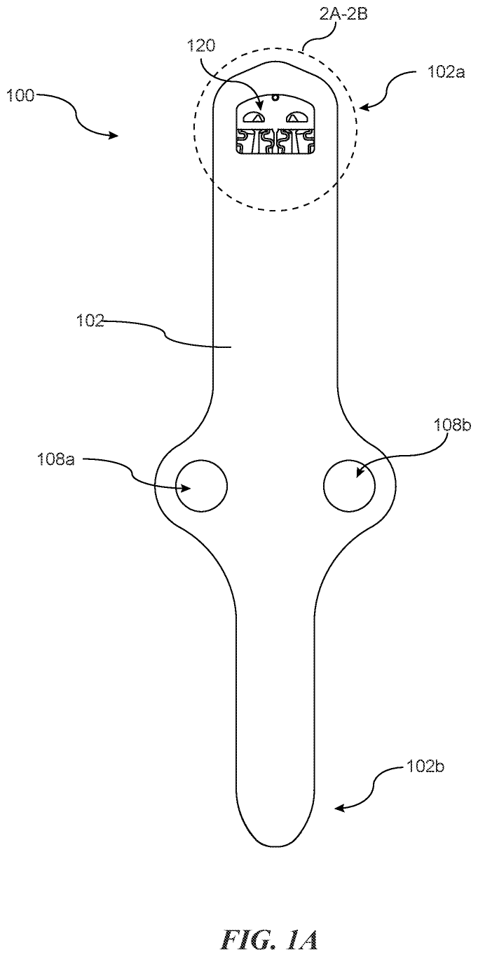

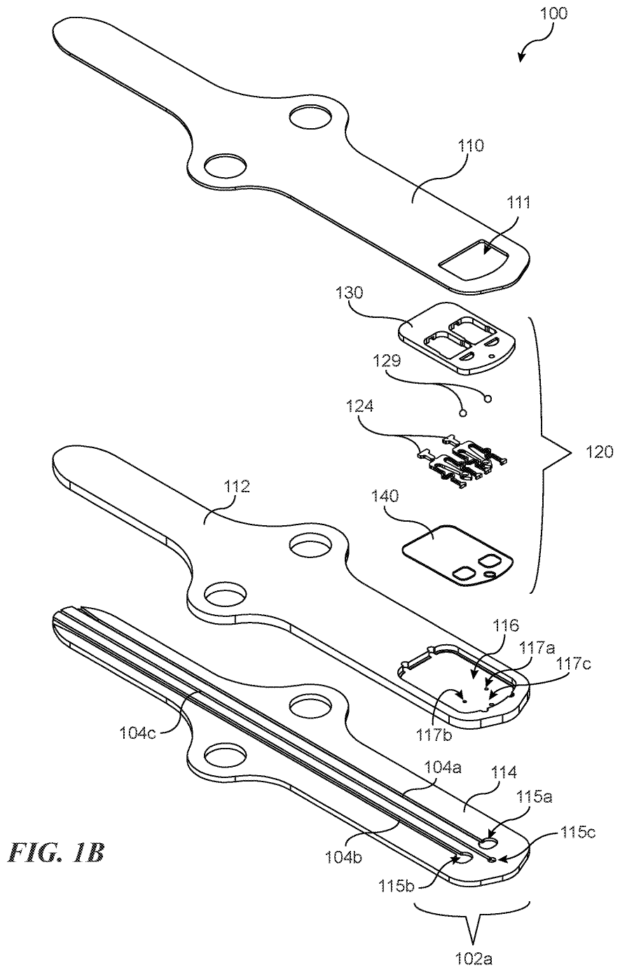

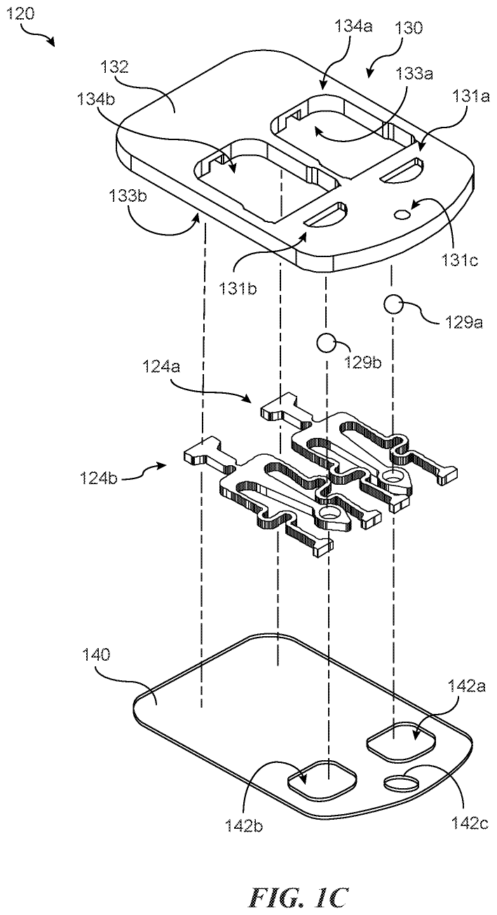

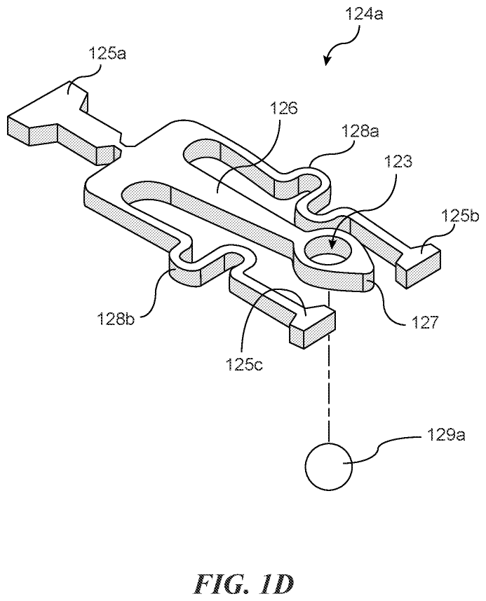

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples and claims but are not described in detail with respect to FIGS. 1A-6B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

The systems described herein can be designed for shunting fluid between a variety of body regions. For example, many of the embodiments described herein are designed to be implanted in a patient's eye to shunt aqueous between the anterior chamber and a target outflow location (e.g., a subconjunctival bleb space), such as to treat glaucoma. However, although certain embodiments are described in terms of shunting fluid from an anterior chamber of an eye, one of skill in the art will appreciate that the present technology can be readily adapted to shunt fluid from and/or between other portions of the eye or, more generally, from and/or between a first body region and a second, different body region of a patient. Moreover, while certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up including, but not limited to, heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

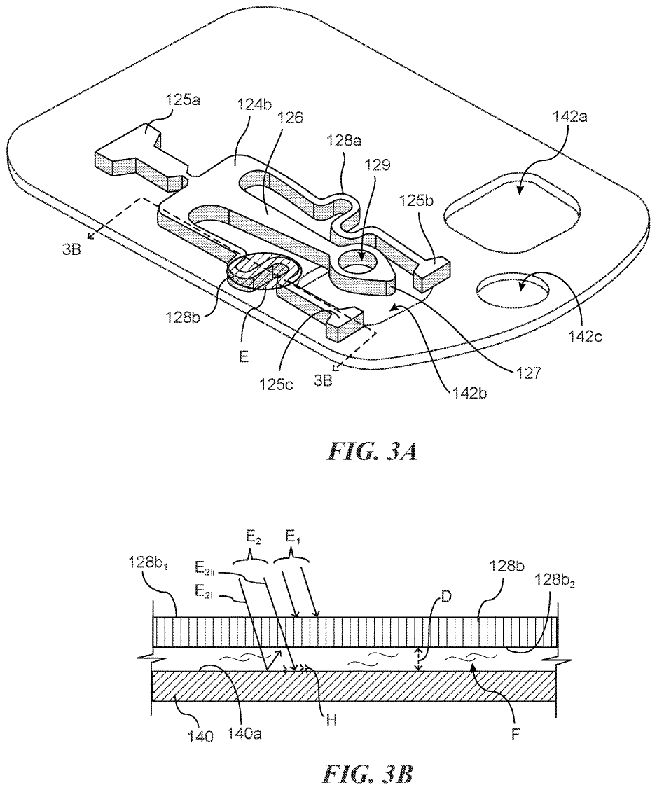

FIGS. 1A-1D illustrate an adjustable shunting system 100 (“the system 100”) configured in accordance with select embodiments of the present technology. More specifically, FIG. 1A is a top view of the system 100, FIG. 1B is an exploded perspective view of the system 100, FIG. 1C is an enlarged view of an actuation assembly 120 of the system 100 as shown in FIG. 1B, and FIG. 1D is an enlarged view of a first actuator 124a of the actuation assembly 120. As described in greater detail below, the system 100 is configured to be adjustable in response to laser energy input to provide a titratable therapy for shunting fluid from a first body region to a second body region, such as shunting aqueous from an anterior chamber of a patient's eye to a target outflow location. As also described in detail throughout this Detailed Description, particularly with reference to FIGS. 3A and 3B, the system 100 can include certain features that are expected to improve the operation of such laser-adjustable shunts, such as by improving the efficiency and/or safety of using laser energy to selectively adjust the system 100 after implantation within the patient.

Referring first to FIG. 1A, the system 100 includes a shunting element 102 and an actuation or control assembly 120, which is partially hidden from view by the shunting element 102 in FIG. 1A. The shunting element 102 (which can also be referred to as a casing, membrane, elongated housing, or the like) extends between a first end portion 102a and a second end portion 102b. A plurality of flow channels 104 (not visible in FIG. 1A—see FIG. 1B) can extend through the shunting element 102 at least partially between the first end portion 102a and the second end portion 102b. As described in greater detail below, when the system 100 is implanted within a patient between a first body region and a second body region, fluid can flow from the first body region to the second body region via the flow channels. The shunting element 102 may optionally include one or more features to facilitate anchoring the system 100 to patient tissue, such as first and second suture holes 108a, 108b. The shunting element 102 can be composed of a partially flexible and/or biocompatible material, such as silicone, polydimethylsiloxane (PDMS), polymethylmethacrylate (PMA), or the like. For example, the shunting element 102 may be composed of a material having a durometer of between about 60 and about 90, or between about 70 and 80, or about 75. Additional features of shunting elements suitable for use with the present technology are described in International Patent Application No. PCT/US2022/037747, the disclosure of which is incorporated by reference herein in its entirety and for all purposes.

The actuation assembly 120 can be positioned at or proximate the first end portion 102a of the shunting element 102. As described in greater detail below, the actuation assembly 120 can have one or more features that selectively control the flow of fluid through one or more of the flow channels extending through the shunting element 102. In this way, the actuation assembly 120 can be selectively manipulated by a clinician to adjust the resistance through the system 100, and thus the level of therapy provided by the system 100. As also described in greater detailed below, the actuation assembly 120 can have one or more features that act as a physical barrier or shield configured to at least partially block, absorb, and/or redirect (e.g., reflect) laser energy that enters the actuation assembly 120.

Referring next to FIG. 1B, the shunting element 102 can include one or more components and/or layers that are stacked and sealed together to collectively form the shunting element 102. For example, the shunting element 102 can include a first (e.g., top) layer 110, a second (e.g., middle) layer 112, and a third (e.g., bottom) layer 114. Accordingly, in the illustrated embodiment the shunting element 102 includes three layers, although in other embodiments the shunting element 102 can include more or fewer layers, such as one, two, four, five, six, or more layers. In operation, the first layer 110, the second layer 112, and the third layer 114 are sealed together (e.g., glued, adhered, bonded, etc.) to form the shunting element 102. More specifically, a lower surface of the first layer 110 is sealed to an upper surface of the second layer 112, and a lower surface of the second layer 112 is sealed to an upper surface of the third layer 114. Sealing the layers prevents or at least reduces fluid from leaking through the system 100 between layers. Additional details regarding multi-layered shunting systems are described in International Patent Application No. PCT/US2022/037917, the disclosure of which is incorporated by reference herein in its entirety. In other embodiments, the shunting element 102 is formed from a single, contiguous structure, without the need for sealing a plurality of layers together.

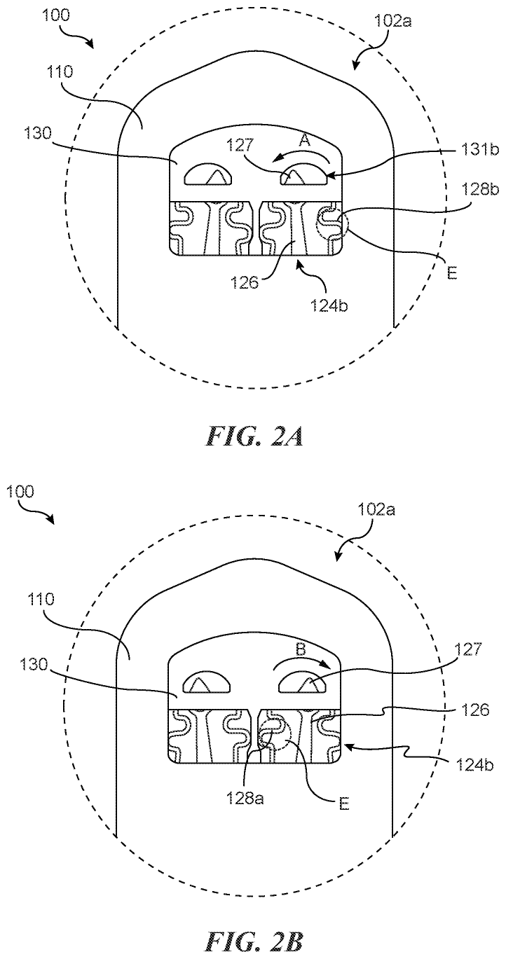

Returning to the embodiment shown in FIG. 1B, the first layer 110 includes an opening 111 that permits fluid to flow into the shunting element 102 (or, depending on the orientation of, and direction of flow through, the shunting element 102, fluid can flow out of the opening 111). In addition to permitting fluid to flow into the shunting element 102, the opening 111 enables a user to have an unobstructed view of at least a portion of the actuation assembly 120. For example, when the system 100 is assembled as shown in FIG. 1A, a portion of the actuation assembly 120 can be aligned with the opening 111. As described in greater detail with reference to FIGS. 2A and 2B, this enables a user to selectively actuate the actuation assembly by directing energy (e.g., laser energy) through the opening 111. Although shown as having a single opening, in some embodiments the first layer 110 may have two more openings.

The second layer 112 includes a chamber or cavity 116 at the first end portion 102a, with an opening to the chamber 116 facing toward the first layer 110. The chamber 116 provides an empty space or cavity for receiving the actuation assembly 120. The chamber 116 also includes several openings (e.g., ports, apertures, etc.). For example, the chamber 116 includes a first aperture 117a, a second aperture 117b, and a third aperture 117c (collectively referred to as the apertures 117). The apertures 117 extend fully through the second layer 112 such that fluid can flow through the second layer 112 via the apertures 117.

The third layer 114 defines or at least partially defines the flow channels that extend through the shunting element 102. In the illustrated embodiment, for example, the third layer 114 defines a first channel 104a, a second channel 104b, and a third channel 104c (collectively referred to as the channels 104). More specifically, a void space of the channels 104 can be formed within the third layer 114, with the second layer 112 forming a “top” of the channels 104 (e.g., the channels 104 become closed off once the second layer 112 is sealed to the third layer 114). The third layer 114 also defines a first well 115a fluidly coupled to the first channel 104a at the first end portion 102a, a second well 115b fluidly coupled to the second channel 104b at the first end portion 102a, and a third well 115c fluidly coupled to the third channel 104c at the first end portion 102a. The first well 115a is aligned with, and therefore configured to receive fluid from, the first aperture 117a of the second layer 112. Likewise, the second well 115b is aligned with, and therefore configured to receive fluid from, the second aperture 117b of the second layer 112, and the third well 115c is aligned with, and therefore configured to receive fluid from, the third aperture 117c.

The channels 104 can be designed to provide different fluid resistances. For example, each of the channels 104 may have a different cross-sectional dimension (e.g., cross-sectional area, diameter, circumference, perimeter, etc.) and/or length. For example, the first channel 104a may have a first cross-sectional area, the second channel 104b may have a second cross-sectional area greater than the first cross-sectional area, and the third channel 104c may have a third cross-sectional area that is greater than the first cross-sectional area but less than the second cross-sectional area. If the length of the channels 104 are the same in such embodiments, the first channel 104a has the highest fluid resistance, the second channel 104b has the lowest fluid resistance, and the third channel 104c has an intermediate fluid resistance. The foregoing is provided by way of example only-a person skilled in the art will appreciate that the channels 104 can be designed with any combination of cross-sectional dimensions and lengths to achieve desired relative resistances.

The actuation assembly 120 includes one or more actuators 124, one or more sealing elements 129, a first plate 130, and a second plate 140. More specifically, and as best shown in FIG. 1C, the actuation assembly 120 includes a first actuator 124a and a second actuator 124b (collectively referred to as the actuators 124). Referring to FIGS. 1B and 1C together, the first actuator 124a can be configured to selectively control the fluid resistance and/or flow of fluid through the first aperture 117a of the second layer 112 (and thus through the first channel 104a), and the second actuator 124b can be configured to selectively control the fluid resistance and/or the flow of fluid through the second aperture 117b of the second layer 112 (and thus through the second channel 104b). The actuation assembly 120 does not include an actuator for controlling the fluid resistance and/or flow through the third aperture 117c of the second layer 112 (and thus through the third channel 104c). Rather, the third channel 104c is designed to be “always open” such that it permits at least some degree of fluid flow through the system 100 even when both the first channel 104a and the second channel 104b are blocked/closed. As one skilled in the art will appreciate from the disclosure herein, the present technology is not limited to particular combinations of “always open” and adjustable channels, and can include more or fewer of each channel type. Similarly, although described as having two actuators 124, the system 100 can have more or fewer actuators, such as one, three, four, or more.

The first plate 130 and the second plate 140 are configured to collectively form a cartridge that houses the actuators 124. That is, the first plate 130 and the second plate 140 can be coupled together (via glue, welding, chemical bonding, mechanical connections, or other attachment mechanisms) to form a cassette having an interior that is shaped to house the actuators 124. In such embodiments, the first plate 130 defines a “top” and “sides” of the actuation assembly 120, and the second plate 140 defines a “bottom” of the actuation assembly 120. In other embodiments, the cartridge can be rotated 180 degrees such that the first plate 130 defines the “bottom” and the second plate 140 defines the “top” of the actuation assembly. As described below, the cartridge forms part of the fluid path through the system 100 (e.g., fluid flows through the cartridge as it flows through the system 100). As also described below, the second plate 140 acts as an at least partially absorptive and/or reflective shield or barrier for blocking and/or redirecting laser energy that is directed into an interior of the cartridge to heat, and therefore actuate, the actuators 124.

As described in greater detail below, the first plate 130 can include features configured to hold and “prime” the actuators 124 when the actuation assembly 120 is in an assembled configuration. For example, the first plate 130 can include a first actuator chamber 133a with first anchoring element retention features (not shown) configured to receive the first actuator 124a, including corresponding anchoring elements of the first actuator 124a (see anchoring elements 125 in FIG. 1D). The first plate 130 can also include a second actuator chamber 133b configured to receive and prime the second actuator 124b (collectively referred to as the actuator chambers 133; the openings to the actuator chambers 133 are facing downwardly toward the actuators 124 in the orientation shown in FIG. 1C). The actuator chambers 133 can be sized and shaped such that they at least partially deform (e.g., stretch, tension, compress, etc.) the actuators 124 when the actuators 124 are positioned therein. For example, in some embodiments positioning the anchoring elements of the actuators 124 within corresponding anchoring element retention features in the first plate 130 can increase a length of the actuation elements 128 (e.g., tension) relative to their preferred geometries. In other embodiments, positioning the anchoring elements 125 within corresponding anchoring element retention features can decrease a length of the actuation elements 128 (e.g., compress) relative to their preferred geometries. In embodiments in which the actuators 124 are composed of a shape memory material as described below, this deformation primes the actuators 124 and permits them to be subsequently actuated. Additional details regarding loading and deforming shape memory actuators are described in U.S. Patent Application Publication No. 2021/0251806, previously incorporated by reference herein, and International Patent Application No. PCT/US21/49140, the disclosure of which is incorporated by reference in its entirety and for all purposes.

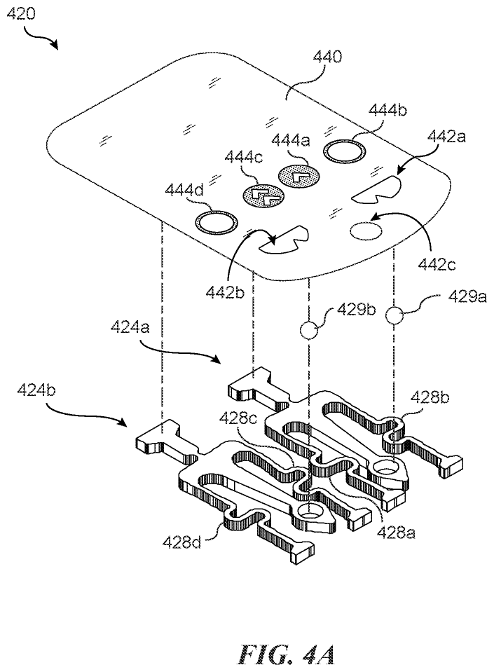

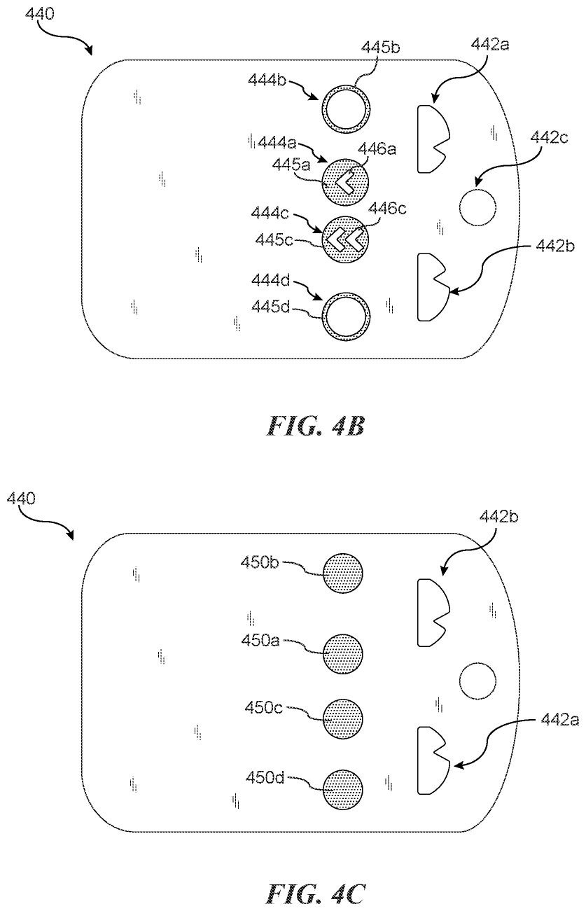

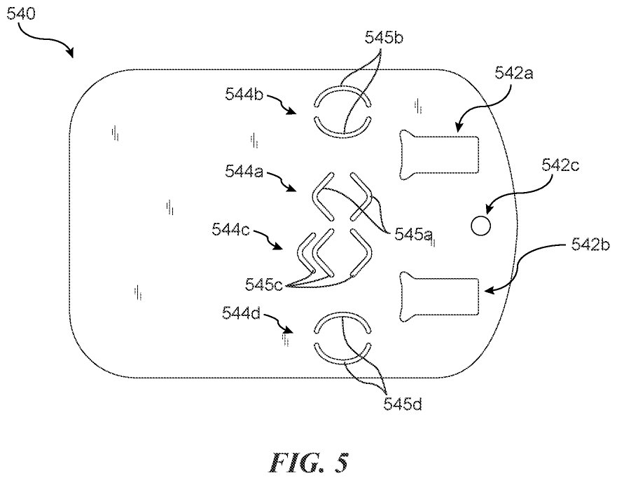

In the illustrated embodiment, the actuator chambers 133 are not fully enclosed via the first plate 130. Rather, the first actuator chamber 133a has a first window 134a that extends through the first plate 130 and aligns with the opening 111 in the first layer 110 of the shunting element 102 (FIG. 1B), and the second actuator chamber 133b has a second window 134b that also extends through the first plate 130 and aligns with the opening 111 in the first layer 110 of the shunting element 102. The first and second windows 134a, 134b are not large enough to permit the actuators 124 from migrating therethrough, but instead enable a user to directly observe at least a portion of the actuators 124. For example, as described in greater detail below, a user can actuate the actuators 124 by directing laser energy through the opening 111 in the first layer 110 of the shunting element and the corresponding first window 134a or the second window 134b of the first plate 130. Although described as first and second “windows,” there need not be a transparent material covering the windows 134, and instead the windows 134 can simply be holes in the first plate 130. Accordingly, when implanted within a fluid environment, fluid can flow through the first window 134a and the second window 134b and into the first actuator chamber 133a and the second actuator chamber 1343b, respectively (e.g., into an interior of the cartridge formed by the first plate 130 and the second plate 140). In other embodiments, such as those described below with reference to FIGS. 4A-5, the windows 134 can be omitted from the first plate 130.

The first plate 130 can further include a first opening 131a, a second opening 131b, and a third opening 131c (collectively referred to as “the first plate openings 131”). Similar to the first window 134a and the second window 134b, the first plate openings 131 permit fluid to flow through the first plate 130 into an interior of the cartridge formed by the first plate 130 and the second plate 140 (e.g., into the first actuator chamber 133a and the second actuator chamber 133b). Additionally, the first opening 131a and the second opening 131b in the first plate 130 can act as visual system-state indicators that enable a user to determine a relative position of the actuators 124 (e.g., a user can see a position of the actuators 124 by looking at the actuators 124 through the first plate openings 131). Additional examples of system-state indicators that can be used with the system 100 are described in International Patent Application No. PCT/US24/13197, the disclosure of which is incorporated by reference herein in its entirety.

The second plate 140 can have a plurality of second plate openings 142. For example, the second plate 140 includes a first opening 142a, a second opening 142b, and a third opening 142c. When the system 100 is assembled, the first opening 142a in the second plate 140 aligns with the first aperture 117a in the second layer 112 of the shunting element 102 (FIG. 1B), the second opening 142b in the second plate 140 aligns with the second aperture 117b in the second layer 112 of the shunting element 102, and the third opening 142c in the second plate 140 aligns with the third aperture 117c in the second layer 112 of the shunting element 102. Accordingly, the second plate openings 142 permit fluid to flow out of the interior of the cartridge formed by the first plate 130 and the second plate 140 and into the channels 104 via the apertures 117. Also, when the system 100 is assembled, a moveable portion of one of the actuators 124 (e.g., a distal end portion 127 of a gating element 126; shown in FIG. 1D) is aligned with the first opening 142a in the second plate 140 and proximate to the first aperture 117a in the second layer 112 of the shunting element 102, and can therefore control the flow of fluid through the first aperture 117a. A moveable portion of the other of the actuators 124 can similarly be positioned to align with, and control fluid flow through, the second aperture 117b.

The first plate 130 and the second plate 140 can be composed of a biocompatible material that has generally stiffer mechanical properties than the layers 110, 112, 114, and/or the actuators 124. For example, the first plate 130 and/or the second plate 140 can be composed of superelastic Nitinol, stainless steel, titanium, a plastic or polymer, combinations thereof, or other suitable materials. This is expected to enable the first plate 130 and the second plate 140 to resist deformation when the actuators 124 are deformed and coupled to the plate 122.

The first plate 130 and/or the second plate 140 can also be composed of a material configured to absorb or reflect laser energy. For example, the first plate 130 and/or the second plate 140 can be composed of a “non-transparent” material that does not permit energy within an operational wavelength range of the laser energy to pass through. The first plate 130 and/or the second plate 140 can therefore be non-transparent to electromagnetic energy having a wavelength of between about 400 nm and about 1,300 nm, or between about 400 nm and about 1,000 nm, or between about 500 and about 1,000 nm, or between about 500 nm and about 800 nm, or between about 500 nm and about 750 nm, or between about 550 nm and about 750 nm, or between about 550 nm and about 700 nm. The foregoing ranges are provided by example only-one skilled in the art will appreciate that the first plate 130 and/or the second plate 140 can be optically non-transparent to electromagnetic energy having wavelengths outside of the foregoing ranges, in addition to or in lieu of being optically non-transparent to wavelengths within the foregoing ranges.

In some embodiments, the second plate 140 can include one or more features that affect its absorptive and/or reflective properties. For example, in some embodiments the first plate 130 and/or the second plate 140 can optionally include an absorptive coating to increase the absorptiveness of the material (e.g., to a particular wavelength of laser energy). In other embodiments, the first plate 130 and/or the second plate 140 can optionally include a reflective coating to increase the reflectiveness of the material (e.g., to a particular wavelength of laser energy). In addition to or in lieu of the coatings, the second plate 140 may also have a non-planar surface that increases the surface area of the second plate without increasing a cross-sectional area of the second plate 140. Examples of non-planar surfaces include textured surfaces, rough surfaces, sawtooth surfaces, and the like. As will be described in detail below with reference to FIGS. 3A and 3B, increasing the surface area of the second plate 140 utilizing non-planar surfaces can be advantageous in embodiments in which the second plate 140 is configured to absorb laser energy and conductively heat a fluid surrounding the second plate 140 and the actuators 124.

As described in greater detail below with reference to FIGS. 3A and 3B, configuring the second plate 140 to at least partially absorb and/or reflect laser energy enables the second plate to (1) improve the efficiency with which the actuators 124 are heated via laser energy (e.g., by improving energy transfer), and (2) act as a shield to prevent or at least reduce laser energy from penetrating through the system 100 and potentially damaging anatomical structures. For example, as noted previously, the second plate 140 can reflect or otherwise redirect some or all of the laser energy that is not directly absorbed by the actuators 124 back toward an underside of the actuators 124. As described in detail below with respect to FIGS. 3A and 3B, this is expected to simultaneously heat the actuators 124 from opposing sides (e.g., the actuators 124 are heated from both a top surface and a bottom surface), which is expected to increase the efficiency of heating the actuators 124. As another example, and as also noted previously, the second plate 140 can absorb some or all of the laser energy that is not directly absorbed by the actuators 124 to heat the actuators 124 through a transmission medium (e.g., fluid) surrounding the second plate 140 and the actuators 124. In some embodiments, the second plate 140 may both absorb some laser energy and reflect or redirect some laser energy. Thus, the second plate 140 is expected to reduce the total amount of laser energy that must be delivered to the patient to actuate the actuators 124 by increasing the amount of the delivered laser energy that is ultimately transferred to the actuators 124 as heat.

As described in greater detail below with reference to FIGS. 2A and 2B, the first actuator 124a can be selectively moveable between (a) a first (e.g., open) position in which the first actuator 124a does not block or at least does not substantially block, and therefore permits fluid flow through, the first aperture 117a, and (b) a second (e.g., closed or at least partially closed) position in which the first actuator 124a substantially blocks and/or seals, and therefore does not permit flow or at least clinically meaningful flow, through the first aperture 117a. That is, the first actuator 124a imparts a greater fluidic resistance through the first aperture 117a when the first actuator 124a is in the second position relative to when the first actuator 124a is in the first position. Likewise, the second actuator 124b can be selectively moveable between (a) a first (e.g., open) position in which the second actuator 124b does not block or at least does not substantially block, and therefore permits fluid flow through, the second aperture 117b, and (b) a second (e.g., closed or at least partially closed) position in which the second actuator 124b substantially blocks and/or seals, and therefore does not permit flow or at least clinically meaningful flow, through the second aperture 117b.

In some embodiments, the first actuator 124a and/or the second actuator 124b can further be selectively moveable between one or more intermediate positions between the first (e.g., open) position and the second (e.g., closed) position. The one or more intermediate positions can provide a different fluid resistance through the corresponding aperture 117. Alternatively, the system 100 can include additional apertures 117 (not shown) such that each actuator 124 is associated with controlling flow through two apertures. In such embodiments, each actuator may be selectively moveable between (a) a first position in which the actuator 124 blocks a first aperture but does not block a second aperture, (b) a second position in which the actuator 124 blocks the second aperture but does not block the first aperture, and (c) a third position in which the actuator 124 does not block the first aperture or the second aperture. In some embodiments the third position is associated with an intermediate position of the actuator 124 that is between the first position and the second position. In other embodiments, the third position is associated with an “end-of-range” position of the actuator 124 that is achieved by actuating only one of the actuation elements, and the intermediate position achieved by actuating both of the actuation elements blocks one of the two apertures. In yet other embodiments, each actuator is associated with controlling flow through three apertures, with each of the three positions associated with blocking one of the three apertures.

Each actuator 124 can also include a sealing element coupled thereto, which are shown separately from the actuators 124 in the exploded view of FIGS. 1B and 1C for ease of illustration. More specifically, as shown in FIG. 1C, the first actuator 124a includes a first sealing element 129a and the second actuator 124b includes a second sealing element 129b (collectively referred to as the sealing elements 129). The sealing elements 129 can be composed of a generally noncompressible material such as glass, plastic, stainless steel, or the like. In other embodiments, the sealing elements 129 can be composed of a partially elastic material, such as silicone, rubber, or the like. Without intending to be bound by theory, the sealing elements 129 are expected to improve the fluid blocking effect (e.g., seal) of the actuators 124 at the corresponding first aperture 117a and second aperture 117b when the actuators 124 are in the closed position. Additional details regarding sealing elements and associated mechanisms for improving a sealing effect that can be used with the system 100 are described in International Patent Application Nos. PCT/US23/20973 and PCT/US23/78333, the disclosures of which are incorporated by reference herein in their entireties.

FIG. 1D is an enlarged view of the first actuator 124a, with other aspects of the system 100 omitted for purposes of illustration. Although FIG. 1D illustrates only the first actuator 124a, one skilled in the art will appreciate that the second actuator 124b can be the same as, or at least generally similar to, the first actuator 124a, and so the description below of the first actuator 124a applies to the second actuator 124b (and any additional actuators) as well.

As shown, the first actuator 124a includes a projection or gating element 126 having a distal end portion 127 configured to at least partially control (e.g., gate) flow through the system 100. To do so, the distal end portion 127 includes a sealing element retention feature 123 configured to hold and retain the first sealing element 129a (the first sealing element 129a is shown removed from the sealing element retention feature 123 in FIG. 1D for purposes of illustration). In the illustrated embodiment, the sealing element retention feature 123 is an annulus, although other suitable configurations are possible and within the scope of the present technology.

The first actuator 124a further includes a first actuation element 128a and a second actuation element 128b. The first actuation element 128a can be configured to rotate, pivot, slide, or otherwise move the gating element 126, and thus the first sealing element 129a, in a first direction. For example, when actuated, the first actuation element 128a can be configured to move the gating element 126 from the second (e.g., closed) position to and/or toward the first (e.g., open) position. The second actuation element 128b can be configured to selectively rotate, pivot, slide, or otherwise move the gating element 126, and thus the first sealing element 129a, in a second direction generally opposite the first direction. For example, when actuated, the second actuation element 128b can be configured to move the gating element 126 from the first (e.g., open) position to and/or toward the second (e.g., closed) position. An example of the operation of moving the gating element 126 between the first and second positions are described below with reference to FIGS. 2A and 2B.

In some embodiments, the first actuation element 128a and the second actuation element 128b (and the first actuator 124a in its entirety) can be composed at least partially of a shape memory material or alloy such as Nitinol. Accordingly, the first actuation element 128a and the second actuation element 128b can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the first actuation element 128a and the second actuation element 128b may have reduced (e.g., relatively less stiff) mechanical properties that cause the actuation elements to be more easily deformable (e.g., compressible, expandable, etc.) relative to when the actuation elements are in the first material state. In the second material state, the first actuation element 128a and the second actuation element 128b may have increased (e.g., relatively more stiff) mechanical properties relative to the first material state, causing an increased preference toward a specific preferred geometry (e.g., original geometry, manufactured or fabricated geometry, heat set geometry, etc.).

The first actuation element 128a and the second actuation element 128b can be selectively and independently transitioned between the first material state and the second material state by applying energy (e.g., laser energy delivered from an energy source external to the system 100 and a patient in which the system 100 is implanted) to the first actuation element 128a or the second actuation element 128b to heat the corresponding actuation element above a transition temperature (e.g., above an austenite finish (Af) temperature, which is generally greater than body temperature). If the first actuation element 128a (or the second actuation element 128b) is deformed relative to its preferred geometry when heated above the transition temperature, the first actuation element 128a (or the second actuation element 128b) will move to and/or toward its preferred geometry. Moving toward its preferred geometry may cause the gating element 126 to move toward the corresponding position (e.g., actuating the first actuation element 128a can move the gating element toward the first (e.g., open) position and actuating the second actuation element 128b can move the gating element toward the second (e.g., closed) position). In some embodiments, the first actuation element 128a and the second actuation element 128b are operably coupled such that, when the actuated actuation element (e.g., the first actuation element 128a) transitions toward its preferred geometry, the non-actuated actuation element (e.g., the second actuation element 128b) is further deformed relative to its preferred geometry. In some embodiments, simultaneously heating both the first actuation element 128a and the second actuation element 128 above their transition temperatures can cause the actuator 128 to assume an intermediate position as both the first actuation element 128a and the second actuation element 128b attempt to assume their preferred geometry and reach an equilibrium. Additional details regarding, and examples of, bi-directional shape memory actuators that can be used with the present technology are described in U.S. Patent Application Publication Nos. US 2020/0229982 and US 2021/0251806 and International Patent Application Publication No. WO 2024/026397, the disclosures of which are incorporated by reference herein in their entireties and for all purposes.

The first actuator 124a further includes a first anchoring element 125a, a second anchoring element 125b, and a third anchoring element 125c (collectively referred to as the anchoring elements 125). As described below, the first actuator 124a can be coupled to the system 100 by placing the anchoring elements 125 in corresponding anchoring element retention features within the first plate 130 of the actuation assembly 120 (FIG. 1C). In some embodiments, the act of placing the anchoring elements 125 in the anchoring element retention features deforms to the actuation elements 128 relative to their preferred geometry, thereby inducing strain in the actuation elements 128 and priming the first actuator 124a for subsequent actuation.

In operation, and as set forth above, the actuation assembly 120 enables a user to selectively change a fluid resistance through, and thus a therapy level provided by, the system 100. For example, FIGS. 2A and 2B illustrate an exemplary operation of changing a fluid resistance through the system 100 in accordance with embodiments of the present technology. More specifically, FIG. 2A illustrates actuating the second actuator 124b to move the second actuator 124b from the first (e.g., open) position toward the second (e.g., closed) position, and FIG. 2B illustrates actuating the second actuator 124b to move the second actuator 124b from the second (e.g., closed) position toward the first (e.g., open) position.

Referring first to FIG. 2A, to move the second actuator 124b toward the second (e.g., closed) position, laser energy E is directed at the second actuation element 128b. The laser energy E heats the second actuation element 128b. As the second actuation element 128b is heated above its transition temperature, the second actuation element 128b transitions toward its preferred geometry. In the illustrated embodiment, the second actuation element 128b is under tension (e.g., lengthened relative to its preferred geometry), and so heating the second actuation element 128b above its transition temperature causes the second actuation element 128b to contract. This causes the distal end portion 127 of the gating element 126 to rotate, pivot, or otherwise move in a first direction (e.g., pivots in a counterclockwise direction), as indicated by the arrow A. This movement moves the sealing element 129b (not visible in FIG. 2A) from the first position in which it does not block, or at least does not substantially block, the second aperture 117b (also not visible in FIG. 2A) toward the second position in which it does at least partially block the second aperture 117b, thereby increasing the fluid resistance through the second aperture 117b and decreasing flow through the second channel 104b (FIG. 1B).

To reverse this operation (i.e., to move the second actuator 124b back toward the first (e.g., open) position, laser energy E is directed at the first actuation element 128a as shown in FIG. 2B. As the first actuation element 128a is heated above its transition temperature, the first actuation element 128a transitions toward its preferred geometry. As with the second actuation element 128b, the first actuation element 128a is under tension in the illustrated embodiment, and so heating the first actuation element 128a above its transition temperature causes the first actuation element 128a to contract. As the first actuation element 128a contracts, the distal end portion 127 of the gating element 126 rotates, pivots, or moves in a second direction (e.g., pivots in a clockwise direction) that is generally opposite to the first direction, as indicated by the arrow B. This movement can move the sealing element 129b from the second position in which it at least partially blocks the second aperture 117b toward the first position in which it does not block the second aperture 117b, thereby decreasing fluid resistance through the second aperture 117b and increasing fluid flow through the second channel 104b (FIG. 1B).

In some embodiments, the second actuator 124b can be moved toward an intermediate position between the first (e.g., open) position and the second (e.g., closed) position by simultaneously directing the laser energy E at both the first actuation element 128a and the second actuation element 128b. This can be accomplished, for example, by using a laser having a larger spot size than shown in FIGS. 2A and 2B. In such embodiments, the laser energy E heats both the first actuation element 128a and the second actuation element 128b above their transition temperatures, causing both the first actuation element 128a and the second actuation element 128b to transition toward their preferred geometries. However, because the first actuation element 128a and the second actuation element 128b work in opposition and therefore the preferred geometries for each generally cannot simultaneously be achieved, the second actuator 124b will reach an equilibrium at a third position between the first position and the second position. In some embodiments, the sealing element 129b may at least partially block the second aperture 117b in the intermediate position. In embodiments in which the second actuator 124b is associated with two apertures 117, the sealing element may unblock both apertures 117 (not shown) when in the intermediate position, as described above.

Although the foregoing operation describes operation of the first actuation element 128a and the second actuation element 128b under tension, one skilled in the art will appreciate that the actuators 124 can be designed such that the actuation elements operate under compression. Similarly, although the foregoing describes actuating the second actuator 124b and thus controlling flow through the second aperture 117b and the second channel 104b, the first actuator 124a can be operated in a similar fashion to control flow through the first aperture 117a and the first channel 104a. Additional detail regarding adjustable shunting systems are described in U.S. Patent Application Publication Nos. US 2020/0229977, US 2020/0229982, US 2021/0251806, US 2022/0142818, and US 2022/0202613, each of which is incorporated by reference herein in its entirety.

As set forth above, the systems described herein are expected to improve the efficiency and/or safety of using laser energy to heat the actuation elements to drive actuation of the system 100. More specifically, the incorporation of a non-transparent, reflective second plate 140 in the actuation assembly 120 is expected to provide several advantages. To illustrate some of these advantages, FIG. 3A provides a perspective view of a portion of the system 100 of FIGS. 1A-1D showing the second actuator 124b and the second plate 140, with other aspects of the system 100 omitted simply for purposes of illustration. FIG. 3B is a cross-sectional view taken along a longitudinal axis of the second actuation element 128b and the second plate 140, along the line shown in FIG. 3A.

Referring first to FIG. 3A, and as described above with reference to FIG. 1C, the second plate 140 is configured to reside “under” the second actuator 124b. As used herein, the relative terms “under” and “below” refers to a direction away from a superficial surface of patient tissue (e.g., away from an outer surface of the patient's eye and/or away from an anterior surface of the patient's eye) when system 100 is implanted in the patient. That is, in embodiments in which the second plate 140 is “under” or “below” the second actuator 124b, the second plate 140 is generally on the opposite side of the second actuator 124b as the source of laser energy (not shown) that is used to actuate the system 100. Thus, it can also be said that the second actuator 124b is “above” the second plate 140. The second plate 140 is likewise under the first actuator 124a, even though the first actuator 124a is omitted from FIG. 3A for ease of illustration. Accordingly, as shown in FIG. 1B, a first (e.g., upper) surface 128b₁ of the second actuation element 128b faces away from the second plate 140 and a second (e.g., lower) surface 128b₂ of the second actuation element 128b faces toward the second plate 140.

As also best shown in FIG. 3B, a first (e.g., upper) surface 140a of the second plate 140 can be spaced apart from the lower surface 128b₂ of the second actuation element 128b by a gap. The gap can have a dimension D (e.g., height) of between about 1 μm and 30 μm, or between about 1 μm and 20 μm, or between about 1 μm and about 15 μm, or between about 1 um and about 10 μm, and/or between about 1 μm and about 5 μm. For example, the small gap may have a dimension D of about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, or about 20 μm. The foregoing is provided by way of example only—in other embodiments, the gap can have a dimension D outside the foregoing ranges and values.

The gap can be filled with a fluid F. As described above with reference to FIG. 1C., the interior of the cartridge formed by the first plate 130 and the second plate 140 (in which the second actuator 124b resides) forms part of the fluid flow pathway through the system 100. Thus, the fluid F in the gap can be whatever fluid the system 100 is shunting. Thus, in the context of an adjustable glaucoma shunt, the fluid F can be aqueous. Moreover, although shown and described as having fluid between the lower surface 128b₂ of the second actuation element 128b and an upper surface 140a of the second plate 140, the fluid F may also extend around the sides of, and even above an upper surface 128b₁ of, the second actuation element 128b. Accordingly, the second actuation element 128b (and the second actuator 148b generally) can sit within a bath of the fluid F.

In some embodiments, the gap is part of an enclosed or substantially enclosed chamber that is sized and shaped to surround an actuatable portion of the second actuation element 128b (e.g., the serpentine shaped portion shown in FIG. 3A). Without intending to be bound by theory, and as described below, enclosing or generally enclosing the gap within a chamber is expected to reduce the rate the fluid F within the gap is exchanged with other fluid, which is expected to (1) concentrate heat transfer between the fluid F and the second actuation element 128b at the appropriate section of the second actuation element 128b, and (b) improve the efficiency of the heat transfer by reducing the likelihood that the heated fluid will be replaced with unheated fluid.

As set forth above, the second actuator 124b is actuated by directing laser energy E (from a laser energy source external to the patient) at either the first actuation element 128a or the second actuation element 128b. As shown in FIG. 3A, a spot size of the laser energy E may be sized to cover a total actuatable section of the targeted actuation element, which is the second actuation element 128a in the illustrated embodiment (e.g., the spot size encompasses generally the entirety of the serpentine or boustrophedon-shaped portion of the second actuation element 128a). As a result, the spot size of the laser energy E may be larger than, or may not align fully with, a surface area of the targeted the second actuation element 128b. This enables a larger area of the actuation element 128b to be directly heated using the laser energy E and/or provides an operator with a greater margin for error. As a result, and as best shown in FIG. 3B, a first portion of the laser energy E₁ will be absorbed by (and therefore heat) the upper surface 128b₁ of the second actuation element 128b, but a second portion of the laser energy E₂ will miss the second actuation element 128b and continue on a downward trajectory.

The upper surface 140a of the second plate 140 can be configured to at least partially redirect the second portion of the laser energy E₂ that is not absorbed by the upper surface 128b₁ of the second actuation element 128b (e.g., as shown via the portion of the laser energy labeled E₂ᵢ in FIG. 3B) and/or absorb the second portion of the laser energy E₂ that is not absorbed by the upper surface 128b₁ of the second actuation element 128b (e.g., as shown via the portion of laser energy labeled E₂ᵢᵢ in FIG. 3B). This is expected to be beneficial for several reasons. First, absorbing and/or reflecting unabsorbed laser energy E₂ is expected to increase the safety of using laser energy to actuate the actuation elements 128 of the system 100. In particular, by absorbing and/or reflecting the second portion of the laser energy E₂, the second portion of the laser energy E₂ does not continue on its downward trajectory and thus does not advance beyond the system 100 into structures of the patient's anatomy that could be negatively affected (e.g., damaged) by the laser energy E₂.

Second, reflecting some or all of the laser energy E₂ (as shown in FIG. 3B as the portion of laser energy E₂ᵢ) is also expected to improve the efficiency with which the second actuation element 128b is heated. This is because some or all of the laser energy E₂ is redirected by the second plate 140 toward the lower surface 128b₂ of the second actuation element 128b that is facing the second plate 140. As a result, the lower surface 128b₂ can directly absorb the reflected laser energy E₂ as heat. Thus, by virtue of the second plate 140 reflecting the laser energy E₂, the second actuation element 128b is simultaneously heated from both the upper surface 128bi and the lower surface 128b₂. This configuration is expected to reduce the total amount of energy and/or time needed to heat the second actuation element 128b above its transition temperature.

Further yet, absorbing some or all of the laser energy E₂ (as shown in FIG. 3B as the portion of laser energy E₂ᵢᵢ) is also expected to improve the efficiency with which the second actuation element 128b is heated. This is because any laser energy E₂ that is absorbed by the second plate 140 will heat the second plate 140. As a result, the second plate 140 can conductively heat the fluid F (as shown in FIG. 3B via the heat H), which as described above can conduct the heat to the second actuation element 128b itself. In embodiments in which the plate 140 is configured to absorb the laser energy E₂, the upper surface 140a of the second plate 140 can optionally be non-planar (e.g., roughened, textured, sawtooth, etc.) to increase the surface area. This is expected to increase the area of contact between the upper surface 140a and the fluid F, which is expected to further improve the efficiency with which the fluid F is conductively heated via the second plate 140. The gap may also be part of an enclosed chamber surrounding an actuatable portion of the second actuation element 128, which as described above is expected to retain the heated fluid F at the appropriate position along the second actuation element 128. Moreover, depending on the type of fluid F, the fluid F may conduct heat better than the second actuation element 128b itself, such that a relatively larger volume of the fluid F is heated, which in turn can heat a larger volume of the second actuation element 128b than could be directly heated by the laser energy E. As a result, the second actuation element 128b can be heated with greater efficiency as compared to embodiments without the second plate 140. Accordingly, regardless of whether the second plate 140 is configured to absorb or redirect the laser energy E₂, the second plate 140 is expected to improve the efficiency with which the second actuation element 128b can be heated.

In some embodiments, the second plate 140 can be tuned to absorb electromagnetic energy at a first wavelength and reemit the absorbed electromagnetic energy at a second wavelength, different than the first wavelength (e.g., via the Stokes effect or the Anti-Stokes effect). In such embodiments, the second plate 140 can be tuned such that the second wavelength corresponds to an optimal or near optimal wavelength for heating the actuation element 128b. That is, the second plate 140 may transform one or more properties of the electromagnetic energy to a form that more efficiently heats the actuation element 128b. In some embodiments, the actuation element 128 itself can also be tuned to specific wavelengths. For example, the upper surface 128b₁ of the second actuation element 128 may be tuned to be most efficiently heated by the first wavelength (e.g., the wavelength of the incident laser energy), whereas the lower surface 128b₂ of the second actuation element 128 may be tuned to be most efficiently heated by the second wavelength (e.g., the wavelength of the re-emitted energy).

Additionally, in some embodiments some portion of the laser energy E₂ may directly heat the fluid F by being absorbed by the fluid F (e.g., without first heating the plate 140). Such heating of the fluid F can contribute to heating the second actuation element 128b via a similar mechanism (e.g., conductive heating) as described above with respect to the second plate 140 heating the fluid F.

Although the foregoing describes the effect of the second plate 140 on the actuation of the second actuation element 128b of the second actuator 124b, the second plate 140 can have the same effect during actuation of the first actuation element 128a, as well as during actuation of the first actuator 124a. The second plate 140 may also provide further advantages beyond those expressly identified herein.

In some embodiments, actuation assemblies configured in accordance with the present technology can rely upon conductive heat transfer between a plate and an actuator as the primary modality of heating, and thus actuating, the actuator to change a fluid resistance through an adjustable shunt. For example, FIG. 4A is an exploded view of such an actuation assembly 420 configured in accordance with select embodiments of the present technology. The actuation assembly 420 can be used with the system 100 of FIGS. 1A and 1B instead of the actuation assembly 120 (FIGS. 1C and 1D), and/or with other similar adjustable shunting systems. As shown in FIG. 4A, the actuation assembly 420 includes a first actuator 424a and a second actuator 424b, which can be the same as or generally similar to the first actuator 124a and the second actuator 124b, respectively, of the actuation assembly 120 (FIGS. 1C and 1D). The actuation assembly 420 also includes a first sealing element 429a and a second sealing element 429b, which can be the same as or generally similar to the first sealing element 129a and the second sealing element 129b, respectively, of the actuation assembly 120.

The actuation assembly 420 further includes at least one plate 440. Of note, when the actuation assembly 420 is assembled and positioned within the system, the plate 440 is positioned “above” the first actuator 424a and the second actuator 424b. Consistent with the previously noted definition for the relative term “under,” the relative term “above,” as used herein, refers to a direction toward a superficial surface of patient tissue (e.g., toward the outer surface of the patient's eye and/or toward an anterior surface of the patient's eye), e.g., such that in embodiments in which the plate 440 is “above” the actuators 424, the plate 440 is generally positioned between the actuators 424 and the source of laser energy (not shown) that is used to actuate the actuators 424. The plate 440 can include a plurality of plate openings 642 (shown as a first plate opening 642a, a second plate opening 642b, and a third plate opening 642c) for permitting fluid to flow through the plate 440.

The actuators 424 can operate in a manner generally similar to that described above with reference to the actuators 124 of the actuation assembly 120. For example, the actuators 424 can be composed of a shape memory material and can each be selectively, independently, and repeatedly transitioned through two or more different positions, with each position providing a different fluid resistance through the adjustable shunt. To facilitate this, the first actuator 424a can include a first actuation element 428a and a second actuation element 428b, and the second actuator 424b can include a third actuation element 428c and a fourth actuation element 428d (collectively referred to as “the actuation elements 428”). However, unlike the actuation assembly 120 that includes windows 134 in the first plate 132 positioned “above” the actuators 124 to enable a user to directly target the actuators 124, the actuation assembly 420 does not provide a direct line-of-sight to the actuation elements 428. That is, the plate 440 does not include openings or windows that align with the actuation elements 428, and therefore the plate 440 prevents a user from directly seeing the actuation elements 428.

Instead, the plate 440 includes one or more actuation targets or zones that, when heated by a laser, conductively transfer heat to the actuation elements 428 (e.g., by conductively transferring heat from an upper surface of the plate 440 to a lower surface of the plate 440). In particular, the plate 440 includes a first actuation target or zone 444a, a second actuation target or zone 444b, a third actuation target or zone 444c, and a fourth actuation target or zone 444c (collectively, the “actuation targets 444”). Each actuation target 444 corresponds to a particular actuation element 428. For example, to heat (and therefore actuate) the first actuation element 428a, a user can direct laser energy at the first actuation target 444a, to heat (and therefore actuate) the second actuation element 428b, a user can direct laser energy at the second actuation target 444b, etc. To heat (and therefore actuate) the first actuation element 428a and the second actuation element 428b simultaneously, such as to move the actuator 424 to an intermediate position or state as described previously, a user can simultaneously direct laser energy at both the first actuation target 444a and the second actuation target 444b (e.g., by using a laser having a larger spot size). As described in greater detail below with reference to FIG. 4B, the actuation targets 444 can include etching, markings, or the like that enable a user to quickly and easily determine which actuation target 444 to target to induce a desired adjustment.

In some embodiments, the actuators 424 are configured to directly contact the plate 440 (e.g., an “upper” surface of the actuators 424 directly contacts a “lower” surface of the plate 440). In such embodiments, heat can be directly and conductively transferred between the plate 440 and the actuators 424 (or, more specifically, between the actuation target 444 and the corresponding actuation element 428). In other embodiments, the actuators 424 are spaced apart from the plate 440 by a gap (e.g., the upper surface of the actuators 424 is separated from lower surface of the plate 440 by space). In such embodiments, the actuators 424 are nevertheless proximate to the plate 440. For example, the gap can have a dimension (e.g., height) of less than about 30 μm, such as between about 1 μm and 30 μm, or between about 1 μm and 20 μm, or between about 1 μm and about 15 μm, or between about 1 μm and about 10 μm, and/or between about 1 μm and about 5 μm. Fluid or another transmission medium can occupy the space formed between the actuators 424 and the plate 440 (e.g., the gap), and heat can be conducted through the fluid between the plate 440 and the actuator 424, in a manner similar to that described above with reference to FIG. 3B. Thus, an actuator that is “proximate” to a plate refers to the actuator 424 being within at least about 30 μm of the plate 440, and/or positioned close enough to the plate that heat can be conductively transferred from the plate 440 to the actuators 424 in response to directing laser energy at the plate 440.

FIG. 4B is a top view of the plate 440 and illustrates additional details of the actuation targets 444. In particular, each actuation target 444 includes a surface pattern 445 to help a user identify the actuation target 444. For example, the first actuation target 444a includes a first surface pattern 445a, the second actuation target 444b includes a second surface pattern 445b, etc. The surface patterns 445 can be formed via etching (e.g., laser etching), staining, oxidizing, or otherwise marking select portions of the actuation targets 444.

In some embodiments, the surface patterns 445 can be at least partially different to assist a user in determining which actuation target 444 to heat in order to induce a desired adjustment. For example, the first actuation target 444a, which when targeted actuates the first actuation element 428a of the first actuator 424a (FIG. 4A) and decreases fluid resistance/increase fluid flow, can have a first surface pattern 445a that defines a first visual indicator 446a that indicates that targeting the first actuation target 444a will reduce fluid resistance/increase fluid flow. The first visual indicator 446a is shown as having a chevron shape, although in other embodiments the first visual indicator 446a can be a plus or other suitable shape. The second actuation target 444b, which when targeted actuates the second actuation element 428b of the first actuator 424a (FIG. 4A) and increase fluid resistance/decreases fluid flow, can have a second surface pattern 445b that indicates that targeting the second actuation target 444b will increase fluid resistance/decrease fluid flow. For example, the second surface pattern 445b has an annular shape. Although not shown, in some embodiments the second surface pattern 445b can define a second visual indicator, similar to how the first surface pattern 445a defines the first visual indicator 446a. However, because the second actuation target 444b is associated with increasing fluid resistance/decreasing fluid flow, the second surface pattern could define a minus shape or other shape indicative of reducing fluid flow. The third surface pattern 445c and the fourth surface pattern 445d can similarly define visual indicators that assist a user in quickly and easily determining how fluid resistance/flow will be adjusted if the third actuation target 444c and the fourth actuation target 444d are targeted, respectively. In the illustrated embodiment, the third surface pattern 445c includes a third visual indicator 446c that is different than the first visual indicator 446a of the first actuation target 444a (e.g., double chevron versus single chevron) to indicate that targeting the third actuation target 444c will decrease fluid resistance/increase flow to a greater extent than targeting the first actuation target 444a. Additional details about system state/position indicators that can be used in combination with the present technology are described in International Patent Application No. PCT/US24/13197, the disclosure of which is incorporated by reference herein in its entirety.

The plate 440 can be composed of an at least partially conductive and rigid material such as Nitinol (e.g., superelastic Nitinol), stainless steel, titanium, a plastic or polymer, combinations thereof, or other suitable materials configured to at least partially absorb and/or conduct laser energy as heat. For example, the plate 440 can be composed of a “non-transparent” material that does not permit energy within an operational wavelength range of the laser energy to pass through, such as between about 400 nm and about 1,300 nm, or between about 400 nm and about 1,000 nm, or between about 500 and about 1,000 nm, or between about 500 nm and about 800 nm, or between about 500 nm and about 750 nm, or between about 550 nm and about 750 nm, or between about 550 nm and about 700 nm, or other suitable ranges.

The plate 440 can also include one or more features in addition to the material composition of the plate 440 itself that increase the efficiency with which the actuation targets 444 absorb and/or conductively transfer heat to the corresponding actuation element 428 (FIG. 4A). For example, the actuation targets 444 can be oxidized, coated, colored, or otherwise modified to increase the absorptiveness and/or conductivity of the actuation targets 444. In some embodiments, the actuation targets 444 can include textured surfaces, rough surfaces, sawtooth surfaces, or the like, to increase the absorptiveness of the actuation targets 444.

In some embodiments, one or more features can be positioned between the actuation targets 444 and the actuation elements 428 to increase the efficiency with which heat is conductively transferred to the actuation elements 428. For example, FIG. 4C illustrates a “bottom” surface of the plate 440 (i.e., the plate 440 has been rotated 180 degrees about its longitudinal axis relative to the views in FIGS. 4A and 4B) and shows that the plate 440 can include one or more conductive zones 450. Each conductive zone 450 forms part of the conductive pathway between an individual actuation target 444 and an individual actuation element 428. For example, the first conductive zone 450a forms part of the conductive pathway between the first actuation target 444a and the first actuation element 428a, the second conductive zone 450b forms part of the conductive pathway between the second actuation target 444b and the second actuation element 428b, etc. The conductive zones 450 can be integral with the plate 440, or can be a separate feature coupled to the plate 440. In some embodiments, the conductive zones 450 are formed by depositing one or more layers of conductive material onto the plate 440. Suitable conductive materials include gold or other metals having a higher conductivity than the material the plate 440 is composed of.

Referring collectively to FIGS. 4A-4C, in some embodiments each of the actuation targets 444 is at least partially conductively isolated. Without being bound by theory, this is expected to reduce heating of a non-targeted actuation target 444, and thus a non-targeted actuation element 428, upon heating of an adjacent actuation target 444. In some embodiments, the conductive isolation can be achieved simply by spacing the actuation targets 444 apart by a particular distance, such as by at least 1 mm, at least 2 mm, at least 3 mm, at least 4 mm, etc. In other embodiments, the plate 440 may include one or more insulative regions between the actuation targets 444 to further minimize incidental heating of non-targeted actuation targets 444 or actuation elements 428.

In some embodiments, the actuation assembly 420 can include one or more additional plates. For example, the plate 440 can be a first plate, and the actuation assembly 420 can include a second plate defining one or more actuation chambers for retaining and tensioning the actuation elements 428, similar to the embodiment described above with reference to FIG. 1C. The first plate 440 and the second plate can be coupled together to enclose the actuators 424 as a cartridge or cassette, and positioned within the shunting element 102 (FIGS. 1A and 1B). Such embodiments differ from the actuation assembly 120 in that they are rotated 180 degrees relative to the actuation assembly 120 such that the plate 440 that sits “above” the actuators 424 does not include any actuation windows or openings. In other embodiments, an underside (not visible) of the plate 420 can include one or more features sized and shape to hold and prime the actuators 424, similar to the actuator chambers 133 on the first plate 130, as described above with reference to FIG. 1C. In such embodiments, the plate 440 can form the entire cartridge that houses the actuators 424. That is, in such embodiment the actuation assembly 420 does not include a second plate.

FIG. 5 illustrates an alternative embodiment of a plate 540 that can be used with the actuation assembly 420 in place of the plate 440, and that is configured in accordance with select embodiments of the present technology. As shown, the plate 540 includes a plurality of actuation targets 544 (shown as a first actuation target 544a, a second actuation target 544b, a third actuation target 544c, and a fourth actuation target 544d), each of which corresponds to a particular actuation element 428. However, unlike the actuation targets 444, the actuation targets 544 have patterned through holes 545a-d instead of surface patterns 445. The patterned through holes 545a-d can extend between upper and lower surfaces of the plate 540. The patterned through holes 545a-d can also be sized and shape to indicate to a user what type of adjustment will occur (e.g., increase fluid resistance, decrease fluid resistance) will occur when the actuation target 544 is heated. In operation, some laser energy may pass through the through holes 545a-d, but the majority of heat transfer will still occur via conductive heat transfer through the actuation targets 544.

Without being bound by theory, conductively heating actuators using a plate positioned above the actuators is expected to provide a number of potential advantages. First, the plate provides a large surface for absorbing laser energy, which reduces the likelihood that laser energy may travel beyond the shunting system and into patient tissue. Second, visual markers can be added to the plate to indicate where a physician should point a laser to induce a desired adjustment, which is expected to simplify the process of adjusting the shunting systems and provide increased usability/predictability for desired adjustments. Other advantages may exist as well.

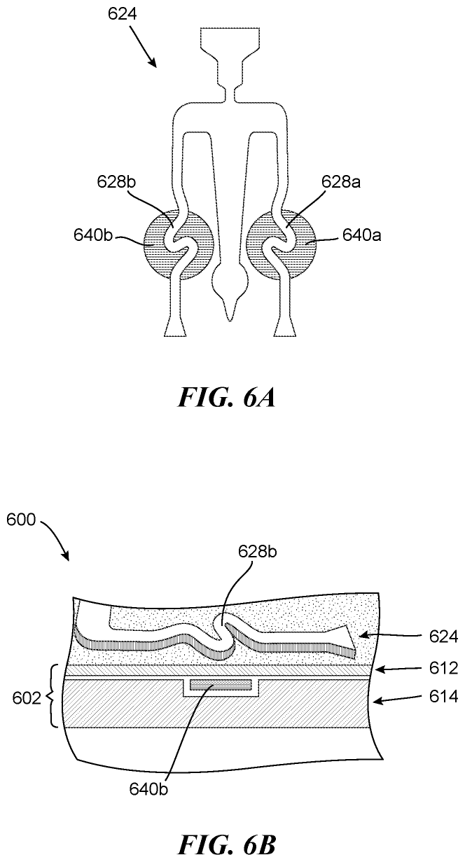

In some embodiments, shunting systems of the present technology can include individual shielding features that function in a manner generally similar to the second plate 140 in the embodiment described with reference to FIGS. 3A and 3B and the plate 440 in the embodiment described with reference to FIGS. 4A-4C. For example, FIG. 6A illustrates an actuator 624 positioned generally above a first shielding element 640a and a second shielding element 640b, and configured in accordance with select embodiments of the present technology. The actuator 624 can be generally similar to or the same as the actuators 124 and 424 described previously, and can include a first actuation element 628a and a second actuation element 628b. The actuatable regions (e.g., the serpentine or boustrophedon-shaped regions) of the actuation elements 628 can be positioned above corresponding shielding features. Specifically, the first actuation element 628a is positioned above a first shield element 640a and the second actuation element 628b is positioned above a second shield element 640b (collectively referred to as the shield elements 640). The shield elements 640 can function generally similar to the plates 140 and 440, described previously. For example, the shield elements 640 can be configured to at least partially block, absorb, and/or redirect laser energy. Accordingly, the shield elements 640 can be composed of any of the materials and have any of the properties described above with reference to the plates 140 and 440. However, relative to the plates 140 and 440, the shield elements 640 have a smaller profile. In particular, the shield elements 640 can have a surface area that is generally similar to or slightly larger than the actuatable region of the actuation elements 628, e.g., as opposed to extending under an entirety or substantial entirety of the actuator 624.

The shield elements 640 can be incorporated into a system for shunting fluid. For example, FIG. 6B is a partially elevated, side cross-sectional view of an adjustable shunting system 600 (“the system 600”). The system 600 can be generally similar to the system 100 described with reference to FIGS. 1A and 1B. For example, the system 600 can include a multi-layered shunting element 602 composed of silicone or another suitable material. As shown in FIG. 6B, the second shield element 640b can be positioned between a first layer 612 and a second layer 614 of the shunting element 602. To facilitate correct placement and retention of the second shield element 640b, the second layer 614 can have a cavity or chamber 615 for receiving and retaining the second shield element 640b. The chamber 615 can be positioned to ensure that the second shield element 640b is aligned with and under the second actuation element 628b. Although not shown, the first shield element 640a (FIG. 6A) can similarly be positioned between the first layer 612 and the second layer 614 and under the first actuation element 628a, e.g., using another cavity or chamber in the second layer 614. In this way, the shield elements 640 can block, absorb, and/or redirect laser energy that is not directly absorbed by the actuator 624.

The systems described herein can be designed for shunting fluid between a variety of body regions. As noted above, for example, in some embodiments the systems described herein are designed to be implanted in a patient's eye to shunt aqueous between the anterior chamber and a target outflow location (e.g., a subconjunctival bleb space), such as to treat glaucoma. Accordingly, in some embodiments the systems described herein can have dimensions compatible with being implanted in the patient's eye. For example, the systems described herein (e.g., the system 100) may have a length of between about 4 mm and about 20 mm, such as between about 4 mm and 15 mm, or between about 4 mm and 12 mm, or between about 6 mm and 10 mm, or about 8 mm. In some embodiments, the layers (e.g., the first layer 110, the second layer 112, and/or third layer 114—FIG. 1B) can have a width or thickness less than about 500 microns, less than about 400 microns, less than about 300 microns, and/or less than about 200 microns. In some embodiments, the diameter of the fluidic channels and corresponding apertures (e.g., the channels 104) may be less than about 100 microns, less than about 75 microns, and/or less than about 50 microns, such as about 35 microns. The foregoing dimensions are provided by way of example only, and other dimensions outside the ranges provided above are possible and included within the scope of the present technology. Indeed, the dimensions of the systems described herein may be designed depending on the type of shunting system (e.g., glaucoma shunt vs. hydrocephalus shunt) and intended recipient (e.g., child vs. adult).

### EXAMPLES

Several aspects of the present technology are set forth in the following examples:

1. An actuation assembly activatable using laser energy and for use with an adjustable shunting system configured to be implanted within a patient, the actuation assembly comprising:
an actuator composed at least partially of a shape memory material, wherein the actuator includes—
   a control element moveable between at least a first position and a second position, and
   an actuation element having a first surface and a second surface opposite to the first surface,
   wherein, in response to being heated, the actuation element is configured to move the control element from the first position toward the second position; and
a plate positioned proximate the actuator such that the second surface of the actuation element faces the plate and the first surface of the actuation element faces away from the plate, wherein the plate is configured such that, in response to the first surface of the actuation element being heated by laser energy delivered from an energy source external to the patient, the plate at least partially redirects a portion of the laser energy not absorbed by the first surface toward the second surface such that both the first surface and the second surface are heated by the laser energy.

2. The actuation assembly of example 1 wherein the plate is configured to at least partially redirect the portion of the laser energy such that both the first surface and the second surface are simultaneously heated by the laser energy.

3. The actuation assembly of example 1 or example 2 wherein the plate is non-transparent.

4. The actuation assembly of any of examples 1-3 wherein the plate is composed at least partially of Nitinol, stainless steel, titanium, and/or plastic.

5. The actuation assembly of any of examples 1-4 wherein the plate is composed at least partially of superelastic Nitinol.

6. The actuation assembly of any of examples 1-5 wherein the plate is spaced apart from the second surface of the actuation element by a gap.

7. The actuation assembly of example 6 wherein the gap has a dimension of between about 1 μm and 20 μm.

8. The actuation assembly of example 6 or example 7 wherein the gap is sized and shaped to receive a fluid, and wherein, during operation when fluid is present, the redirected portion of the laser energy heats the fluid in addition to heating the second surface of the actuation element.

9. The actuation assembly of any of examples 1-8 wherein the plate is a second plate, and wherein the actuation assembly further comprises a first plate, and further wherein:
the first plate and second plate are configured to be coupled together to form a cartridge having an interior, and
the actuator is positioned within the interior of the cartridge.

10. The actuation assembly of example 9 wherein the first plate and the second plate are composed of the same material.

11. The actuation assembly of example 9 or example 10 wherein the first plate and the second plate are both composed of superelastic Nitinol.

12. The actuation assembly of any of examples 9-11 wherein the first plate has a window for permitting ingress of laser energy into an interior of the cartridge.

13. The actuation assembly of any of examples 1-12 wherein the actuation element is configured to transition from a first material state to a second material state in response to being heated above a transition temperature.

14. The actuation assembly of any of examples 1-13 wherein the actuation element is a first actuation element, and wherein the actuator further includes a second actuation element configured to move the control element form the second position toward the first position in response to being heated.

15. The actuation assembly of any of examples 1-14 wherein the actuation assembly is for use with an adjustable glaucoma shunt.

16. An adjustable shunt for shunting fluid from a first body region to a second body region within a patient, the system comprising:
a shunting element having a channel extending therethrough, wherein, when the shunt is implanted in the patient, the shunting element is sized and shaped to extend between the first body region and the second body region;
a laser-activatable actuator for selectively controlling the flow of fluid through the shunting element, the laser-activatable actuator having at least a first surface and a second surface, wherein, in response to being heated, the actuator is transitionable between at least a first position associated with a first fluid resistance through the shunting element and a second position associated with a second fluid resistance through the shunting element; and
a cartridge for housing the actuator, wherein the cartridge includes a plate positioned under the actuator such that the first surface faces away from the plate and the second surface faces the plate, wherein the plate is configured such that, in response to the first surface of the actuation element being heated by laser energy delivered from an energy source external to the patient, the plate at least partially redirects a portion of the laser energy not absorbed by the first surface of the actuator toward the second surface of the actuator such that both the first surface and the second surface are heated by the laser energy.

17. The adjustable shunt of example 16 wherein the plate is a second plate, and wherein the cartridge further includes a first plate positioned over the actuator such that at least a portion of the first surface faces the first plate.

18. The adjustable shunt of example 16 or example 17 wherein an interior of the cartridge is configured to receive a fluid when the shunt is implanted within the patient.

19. The adjustable shunt of any of examples 16-18 wherein the second plate is spaced apart from the second surface by a gap.

20. The adjustable shunt of any of examples 16-19 wherein the plate is non-transparent.

21. The adjustable shunt of any of examples 16-20 wherein the plate is composed at least partially of superelastic Nitinol, and wherein the actuator is composed at least in part of shape memory Nitinol.

22. The adjustable shunt of any of examples 16-21 wherein the plate includes an opening aligned with a corresponding inlet to the channel extending through the shunting element.

23. The adjustable shunt of example 22 wherein the actuator is configured to selectively control the flow of fluid by selectively interfering with the inlet.

24. The adjustable shunt of any of examples 16-23 wherein the plate is configured to at least partially redirect the portion of the laser energy such that both the first surface and the second surface are simultaneously heated.

25. A method of adjusting a shunt having an actuation assembly comprising a shape memory actuator and a plate, the method comprising:
directing laser energy toward an actuation element of the shape memory actuator, wherein the laser energy has a laser spot size larger than a width of a first surface of the actuation element such that a first portion of the laser energy is absorbed by the first surface and a second portion of the laser energy extends beyond the first surface,
wherein the second portion of the laser energy is redirected toward a second surface of the actuation element, opposite the surface, such that the first surface and the second surface are both heated.

26. The method of example 25 wherein the first surface and the second surface are heated simultaneously.

27. The method of example 25 or example 26 wherein the second portion of the laser energy is redirected by reflecting off of a plate positioned under the shape memory actuator.

28. The method of example 27 wherein the plate is non-transparent.

29. The method of example 27 or example 28 wherein the plate is composed of superelastic Nitinol.

30. The method of any of examples 25-29 wherein redirecting the second portion of the laser energy heats fluid surrounding the lower surface of the actuation element in addition to directly heating the second surface of the actuation element.

31. An actuation assembly activatable using laser energy and for use with an adjustable shunting system configured to be implanted within a patient, the actuation assembly comprising:
an actuator composed at least partially of a shape memory material, wherein the actuator includes—
   a control element moveable between at least a first position and a second position, and
   an actuation element having a first surface and a second surface opposite to the first surface,
   wherein, in response to being heated, the actuation element is configured to move the control element from the first position toward the second position;
a plate positioned proximate the actuator such that the second surface of the actuation element faces the plate and the first surface of the actuation element faces away from the plate; and
a gap between the plate and the second surface of the actuation element, wherein, when the actuation assembly is implanted within a patient, the gap is sized and shaped to receive a fluid,
wherein the plate is configured such that, in response to the first surface of the actuation element being heated by laser energy delivered from an energy source external to the patient, the plate at least partially absorbs a portion of the laser energy not absorbed by the first surface and conductively heats the fluid in the gap.

32. The actuation assembly of example 31 wherein the plate is non-transparent.

33. The actuation assembly of any of example 31 or example 32 wherein the plate is composed at least partially of Nitinol, stainless steel, titanium, and/or plastic.

34. The actuation assembly of any of examples 31-33 wherein the plate includes an absorptive coating.

35. The actuation assembly of any of examples 31-34 wherein the gap has a dimension of between about 1 μm and 20 μm.

36. The actuation assembly of any of examples 31-35 wherein the plate has a non-planar surface.

37. The actuation assembly of example 36 wherein the non-planar surface is roughened, textured, and/or saw-toothed.

38. The actuation assembly of any of examples 31-37 wherein the gap is part of an enclosed or generally enclosed chamber.

39. An actuation assembly activatable using laser energy and for use with an adjustable shunting system configured to be implanted within a patient, the actuation assembly comprising:
an actuator composed at least partially of a shape memory material, wherein the actuator includes—
   a control element moveable between at least a first position and a second position, and
   an actuation element,
   wherein, in response to being heated, the actuation element moves the control element from the first position toward the second position; and
a plate positioned proximate the actuator,
wherein, in response to absorbing laser energy delivered from a laser energy source external to the patient, the plate is configured to conductively transfer heat to the actuation element.

40. The actuation assembly of example 39 wherein the plate is positioned above the actuator.

41. The actuation assembly of example 39 or example 40 wherein the plate obstructs a view of the actuation element.

42. The actuation assembly of any of examples 39-41 wherein the plate includes an actuation target aligned with the actuation element.

43. The actuation assembly of example 42 wherein the actuation target includes a surface pattern formed via etching, staining, or oxidization.

44. The actuation assembly of example 42 or example 43 wherein the plate includes a conductive zone positioned between the actuation target and the actuator, and wherein the conductive zone is composed of a material having a higher conductivity than the plate.

45. The actuation assembly of any of examples 39-44 wherein the plate is composed at least in part of Nitinol.

46. The actuation assembly of any of examples 39-45 wherein the actuation assembly includes a gap between the plate and the actuation element, and wherein the gap is configured to be occupied by a fluid when the actuation assembly is implanted in the patient, the fluid forming part of the conductive pathway between the actuation assembly and the actuation element.

47. The actuation assembly of any of examples 39-46 wherein the plate includes one or more features sized and shaped to at least retain and partially deform the actuation element.

48. The actuation assembly of any of examples 39-47 wherein the actuation element is a first actuation element, and wherein the actuation includes a second actuation element that, in response to being heated, moves the control element from the second position toward the first position.

49. The actuation assembly of example 48 wherein the plate includes:
a first actuation target that, in response to absorbing laser energy, conductively transfers heat to the first actuation element, and
a second actuation target that, in response to absorbing laser energy, conductively transfers heat to the second actuation element.

50. A method of adjusting a shunt implanted within a patient, the shunt having an actuation assembly comprising a shape memory actuator and a plate, the method comprising:
directing laser energy from a laser energy source external to the patient toward an actuation target on the plate,
wherein the plate absorbs at least some of the laser energy as heat and conductively transfers at least some of the heat to the actuation element, and
wherein the plate is positioned between the laser energy source and the actuation element.

51. The method of example 50 wherein the conductive heat transfer from the plate to the actuation element is the primary source of heating of the actuation element.

52. The method of example 50 or 51 wherein the plate is separated from the actuation element by a fluid filled gap, and wherein conductively transferring the heat to the actuation element includes heating fluid in the fluid filled gap.

53. The method of any of examples 50-52 wherein the actuation element is heated above a transition temperature such that it undergoes a material composition phase change.

54. The method of any of examples 50-53 wherein the actuation target includes a surface pattern formed via etching, staining, or oxidization.

55. The method of any of examples 50-54 wherein the plate blocks a view of the actuation element.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. An actuation assembly activatable using laser energy and for use with an adjustable shunting system configured to be implanted within a patient, the actuation assembly comprising:
an actuator composed at least partially of a shape memory material, wherein the actuator includes—
a control element moveable between at least a first position and a second position, and
an actuation element having a first surface and a second surface opposite to the first surface,
wherein, in response to being heated, the actuation element is configured to move the control element from the first position toward the second position; and
a plate positioned proximate the actuator such that the second surface of the actuation element faces the plate and the first surface of the actuation element faces away from the plate, wherein the plate is configured such that, in response to the first surface of the actuation element being heated by laser energy delivered from an energy source external to the patient, the plate at least partially redirects a portion of the laser energy not absorbed by the first surface toward the second surface such that both the first surface and the second surface are heated by the laser energy.

2. The actuation assembly of claim 1 wherein the plate is configured to at least partially redirect the portion of the laser energy such that both the first surface and the second surface are simultaneously heated by the laser energy.

3. The actuation assembly of claim 1 wherein the plate is non-transparent.

4. The actuation assembly of claim 1 wherein the plate is composed at least partially of Nitinol, stainless steel, titanium, and/or plastic.

5. The actuation assembly of claim 1 wherein the plate is composed at least partially of superelastic Nitinol.

6. The actuation assembly of claim 1 wherein the plate is spaced apart from the second surface of the actuation element by a gap.

7. The actuation assembly of claim 6 wherein the gap has a dimension of between about 1 μm and 20 μm.

8. The actuation assembly of claim 6 wherein the gap is sized and shaped to receive a fluid, and wherein, during operation when fluid is present, the redirected portion of the laser energy heats the fluid in addition to heating the second surface of the actuation element.

9. The actuation assembly of claim 1 wherein the plate is a second plate, and wherein the actuation assembly further comprises a first plate, and further wherein:
the first plate and second plate are configured to be coupled together to form a cartridge having an interior, and
the actuator is positioned within the interior of the cartridge.

10. The actuation assembly of claim 9 wherein the first plate and the second plate are composed of the same material.

11. The actuation assembly of claim 9 wherein the first plate and the second plate are both composed of superelastic Nitinol.

12. The actuation assembly of claim 9 wherein the first plate has a window for permitting ingress of laser energy into an interior of the cartridge.

13. The actuation assembly of claim 1 wherein the actuation element is configured to transition from a first material state to a second material state in response to being heated above a transition temperature.

14. The actuation assembly of claim 1 wherein the actuation element is a first actuation element, and wherein the actuator further includes a second actuation element configured to move the control element form the second position toward the first position in response to being heated.

15. The actuation assembly of claim 1 wherein the actuation assembly is for use with an adjustable glaucoma shunt.

16. An adjustable shunt for shunting fluid from a first body region to a second body region within a patient, the system comprising:
a shunting element having a channel extending therethrough, wherein, when the shunt is implanted in the patient, the shunting element is sized and shaped to extend between the first body region and the second body region;
a laser-activatable actuator for selectively controlling the flow of fluid through the shunting element, the laser-activatable actuator having at least a first surface and a second surface, wherein, in response to being heated, the actuator is transitionable between at least a first position associated with a first fluid resistance through the shunting element and a second position associated with a second fluid resistance through the shunting element; and
a cartridge for housing the actuator, wherein the cartridge includes a plate positioned under the actuator such that the first surface faces away from the plate and the second surface faces the plate, wherein the plate is configured such that, in response to the first surface of the actuation element being heated by laser energy delivered from an energy source external to the patient, the plate at least partially redirects a portion of the laser energy not absorbed by the first surface of the actuator toward the second surface of the actuator such that both the first surface and the second surface are heated by the laser energy.

17. The adjustable shunt of claim 16 wherein the plate is a second plate, and wherein the cartridge further includes a first plate positioned over the actuator such that at least a portion of the first surface faces the first plate.

18. The adjustable shunt of claim 16 wherein an interior of the cartridge is configured to receive a fluid when the shunt is implanted within the patient.

19. The adjustable shunt of claim 16 wherein the second plate is spaced apart from the second surface by a gap.

20. The adjustable shunt of claim 16 wherein the plate is non-transparent.

21. The adjustable shunt of claim 16 wherein the plate is composed at least partially of superelastic Nitinol, and wherein the actuator is composed at least in part of shape memory Nitinol.

22. The adjustable shunt of claim 16 wherein the plate includes an opening aligned with a corresponding inlet to the channel extending through the shunting element.

23. The adjustable shunt of claim 22 wherein the actuator is configured to selectively control the flow of fluid by selectively interfering with the inlet.

24. The adjustable shunt of claim 16 wherein the plate is configured to at least partially redirect the portion of the laser energy such that both the first surface and the second surface are simultaneously heated.

25. A method of adjusting a shunt having an actuation assembly comprising a shape memory actuator and a plate, the method comprising:
directing laser energy toward an actuation element of the shape memory actuator, wherein the laser energy has a laser spot size larger than a width of a first surface of the actuation element such that a first portion of the laser energy is absorbed by the first surface and a second portion of the laser energy extends beyond the first surface,
wherein the second portion of the laser energy is redirected toward a second surface of the actuation element, opposite the surface, such that the first surface and the second surface are both heated.

26. The method of claim 25 wherein the first surface and the second surface are heated simultaneously.

27. The method of claim 25 wherein the second portion of the laser energy is redirected by reflecting off of a plate positioned under the shape memory actuator.

28. The method of claim 27 wherein the plate is non-transparent.

29. The method of claim 27 wherein the plate is composed of superelastic Nitinol.

30. The method of claim 25 wherein redirecting the second portion of the laser energy heats fluid surrounding the lower surface of the actuation element in addition to directly heating the second surface of the actuation element.

31. An actuation assembly activatable using laser energy and for use with an adjustable shunting system configured to be implanted within a patient, the actuation assembly comprising:
an actuator composed at least partially of a shape memory material, wherein the actuator includes—
a control element moveable between at least a first position and a second position, and
an actuation element having a first surface and a second surface opposite to the first surface,
wherein, in response to being heated, the actuation element is configured to move the control element from the first position toward the second position;
a plate positioned proximate the actuator such that the second surface of the actuation element faces the plate and the first surface of the actuation element faces away from the plate; and
a gap between the plate and the second surface of the actuation element, wherein, when the actuation assembly is implanted within a patient, the gap is sized and shaped to receive a fluid,
wherein the plate is configured such that, in response to the first surface of the actuation element being heated by laser energy delivered from an energy source external to the patient, the plate at least partially absorbs a portion of the laser energy not absorbed by the first surface and conductively heats the fluid in the gap.

32. The actuation assembly of claim 31 wherein the plate is non-transparent.

33. The actuation assembly of claim 31 wherein the plate is composed at least partially of Nitinol, stainless steel, titanium, and/or plastic.

34. The actuation assembly of claim 31 wherein the plate includes an absorptive coating.

35. The actuation assembly of claim 31 wherein the gap has a dimension of between about 1 μm and 20 μm.

36. The actuation assembly of claim 31 wherein the plate has a non-planar surface.

37. The actuation assembly of claim 36 wherein the non-planar surface is roughened, textured, and/or saw-toothed.

38. The actuation assembly of claim 31 wherein the gap is part of an enclosed or generally enclosed chamber.

39. An actuation assembly activatable using laser energy and for use with an adjustable shunting system configured to be implanted within a patient, the actuation assembly comprising:
an actuator composed at least partially of a shape memory material, wherein the actuator includes—
a control element moveable between at least a first position and a second position, and
an actuation element,
wherein, in response to being heated, the actuation element moves the control element from the first position toward the second position; and
a plate positioned proximate the actuator,
wherein, in response to absorbing laser energy delivered from a laser energy source external to the patient, the plate is configured to conductively transfer heat to the actuation element.

40. The actuation assembly of claim 39 wherein the plate is positioned above the actuator.

41. The actuation assembly of claim 39 wherein the plate obstructs a view of the actuation element.

42. The actuation assembly of claim 39 wherein the plate includes an actuation target aligned with the actuation element.

43. The actuation assembly of claim 42 wherein the actuation target includes a surface pattern formed via etching, staining, or oxidization.

44. The actuation assembly of claim 42 wherein the plate includes a conductive zone positioned between the actuation target and the actuator, and wherein the conductive zone is composed of a material having a higher conductivity than the plate.

45. The actuation assembly of claim 39 wherein the plate is composed at least in part of Nitinol.

46. The actuation assembly of claim 39 wherein the actuation assembly includes a gap between the plate and the actuation element, and wherein the gap is configured to be occupied by a fluid when the actuation assembly is implanted in the patient, the fluid forming part of the conductive pathway between the actuation assembly and the actuation element.

47. The actuation assembly of claim 39 wherein the plate includes one or more features sized and shaped to at least retain and partially deform the actuation element.

48. The actuation assembly of claim 39 wherein the actuation element is a first actuation element, and wherein the actuation includes a second actuation element that, in response to being heated, moves the control element from the second position toward the first position.

49. The actuation assembly of claim 48 wherein the plate includes:
a first actuation target that, in response to absorbing laser energy, conductively transfers heat to the first actuation element, and
a second actuation target that, in response to absorbing laser energy, conductively transfers heat to the second actuation element.

50. A method of adjusting a shunt implanted within a patient, the shunt having an actuation assembly comprising a shape memory actuator and a plate, the method comprising:
directing laser energy from a laser energy source external to the patient toward an actuation target on the plate,
wherein the plate absorbs at least some of the laser energy as heat and conductively transfers at least some of the heat to the actuation element, and
wherein the plate is positioned between the laser energy source and the actuation element.

51. The method of claim 50 wherein the conductive heat transfer from the plate to the actuation element is the primary source of heating of the actuation element.

52. The method of claim 50 wherein the plate is separated from the actuation element by a fluid filled gap, and wherein conductively transferring the heat to the actuation element includes heating fluid in the fluid filled gap.

53. The method of claim 50 wherein the actuation element is heated above a transition temperature such that it undergoes a material composition phase change.

54. The method of claim 50 wherein the actuation target includes a surface pattern formed via etching, staining, or oxidization.

55. The method of claim 50 wherein the plate blocks a view of the actuation element.
